(19) United States
(12) Patent Application Publication
(10) Pub. No.: US 2014/0142279 A1
(43) Pub. Date: May 22, 2014

(54) Method for peptide synthesis

(71) Applicants: Iraj Lalezari, Scarsdale, NY (US); Parviz Lalezari, Scarsdale, NY (US)

(72) Inventors: Iraj Lalezari, Scarsdale, NY (US); Parviz Lalezari, Scarsdale, NY (US)

(21) Appl. No.: 13/987,887

(22) Filed: Sep. 12, 2013

Related U.S. Application Data
(60) Provisional application No. 61/743,888, filed on Sep. 13, 2012.

(51) Int. Cl. C07K 1/06; C07D 498/18; C07C 37/50

(52) U.S. Cl. CPC C07K 1/063; C07C 37/50; C07D 498/18
USPC 530/337; 568/799; 548/435

(57) ABSTRACT

A new method based on the synthesis and use of novel N and C protecting agents. The new N-protecting agent, here referred to as V-Phenol, generates V-protected amino acids and can be successfully applied to all conventional peptide bond formations including active esters, N,N′-dicyclohexylcarbodiimide (DCC) or related dehydrating agents mixed anhydride methods, PC13 and related agents. The new C-protecting agent, here referred to as HONE, can be successfully applied to peptide synthesis as an active ester not only in combination with V-protected amino acids but also with other N-protecting agents such as Cbz, Boc, Fmoc, etc.

### FIELD OF THE INVENTION

The present invention relies upon the prior provisional application 61743888 filed on Sep. 13, 2012. This substitute specification contains no new matter. A novel method for peptide synthesis is described which utilizes standard Laboratory equipment and reagents, does not require solid phase peptide synthesis facilities, and can be applied to synthesis of peptides with various numbers of amino acids, in both small and large quantities with high purity and yield. The new technology is applicable to all known L and D amino acids and their combinations, and can be used for synthesis of biologically active peptides and drugs.

### DESCRIPTION OF THE INVENTION

### V-Phenol (C22H20 O5), MW: 364

V-Phenol offers the following advantages: It has a higher molecular weight as compared with Cbz and Boc, it causes none or minimal racemization, it is easily removable by HCl or Trifluoroacetic acid at room temperature, it resists bases and catalytic hydrogenation, and its preparation does not require the use of Phosgen, a highly toxic reagent. Among several compounds synthesized and tested, 4-Benzyloxy-3-Methoxybenzyloxy-Phenoxy Carbonate (V-Phenol) was found to be the most effective compound. V-Phenol is a derivation of Vanillin, is a crystalline product and is stable at room temperature for many years. The synthesis of V-Phenol is described in Scheme 1.

### Synthesis of V-Phenol

| | |
|---|---|
| Vanillin 136.8 g | (1 Mole) |
| Benzyl Chloride 114 ml | (1.1 Mole) |
| Anhydrous Potassium Carbonate 63 g | (0.456 Mole) |
| Isopropanol 450 ml | |

The mixture was refluxed for 18 hours. To the hot mixture 24 g of Sodium Borohydride was added slowly (to keep the reaction under control). Gradually a semisolid mass was formed, which was kept under reflux for additional 2 hours. After cooling, 1,500 ml cold water was added and stirred to give a crystalline colorless compound: 190 g, (83% yield), MP 62-63° C. The structure was confirmed to be 4-benzyloxy-3-methoxy-benzyl alcohol. The alcohol obtained was dried and dissolved in 1140 ml CH2Cl2 and 100 ml pyridine was added. The mixture was cooled in an ice/salt bath for half an hour and then gradually 125 mL phenylchloroformate was added. After 2 hours of stirring at room temperature, cold water was added to give a crystalline compound after removal of most of CH2Cl2. The preparation was filtered and re-crystallized from toluene to give 299 g of white crystals (82% yield), MP: 118-120° C.

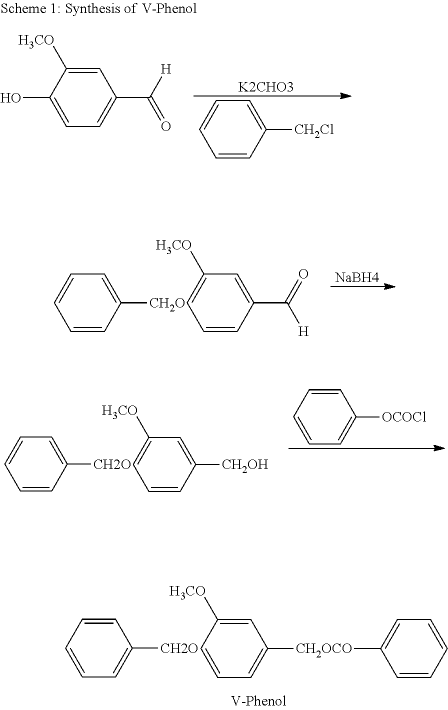

### Synthesis of V-Amino Acids.

V-Phenol reacts with amino acids in mild alkaline solutions in various organic solvents as shown in Scheme 2.

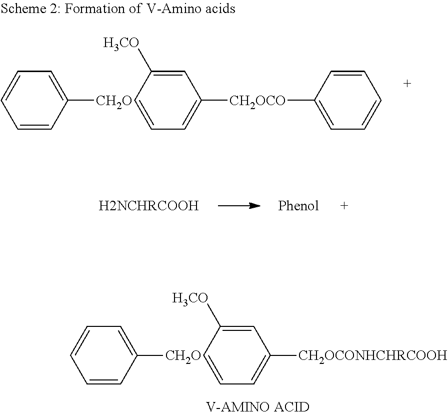

### HONE (C8H7N04), MW=181

HONE, a new C-Protecting active ester, is exo-N-hydroxy-7-oxabycyclo[2.2.1]hept-5-ene-2,3-dicarboximide. It is prepared by Diels-Alder reaction between maleic anhydride and furan in various non-hydroxylic solvents, such as toluene at room temperature. The tricyclic anhydride is reacted with hydroxylamine hydrochloride and mild bases such as sodium bicarbonate or sodium acetate in water. HONE is re-crystallized in water and is stable at room temperature for several years. See Scheme 3.

### Synthesis of HONE

a) 80 g maleic anhydride (2 moles) was dissolves in 350 ml toluene by stirring and gentle warming, cooled to room temperature and then 120 ml of furan was gradually added. After cooling, a crystalline compound (anhydride) was formed. 100% yield. MP=118-120° C.
b) To synthesize HONE a mixture of 320 g anhydride (2 mole), 168 g sodium bicarbonate (2 mole) or equivalent Sodium Acetate, 139 g hydroxylamine hydrochloride and 400 ml of water was warmed at 60-70° C. for one hour and then cooled. Crystals were formed, which were filtered by suction and washed with 50 mL cold water. After drying, 350 g (97%) crystalline compound was obtained. Re-crystallization was done in boiling water to give prisms. MP=202-204° C.

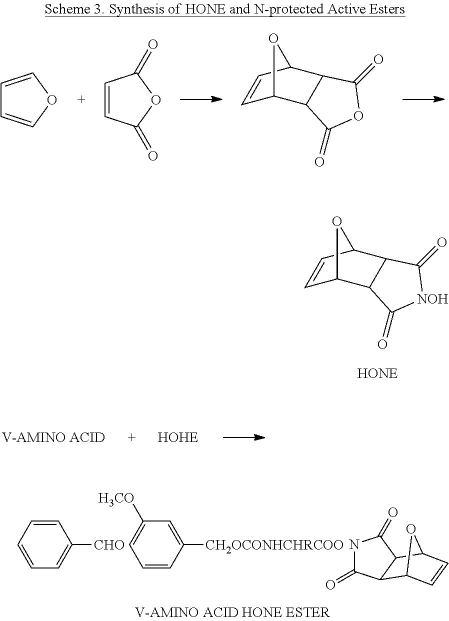

Preparation of N-protected Amino acid Active Esters

N-protected amino acids and HONE are dissolved in non-protic solvents and reacted with dicyclohexylcarbodiimide (DCC) at cold to give N-protected amino acid-HONE esters. See Scheme 3.

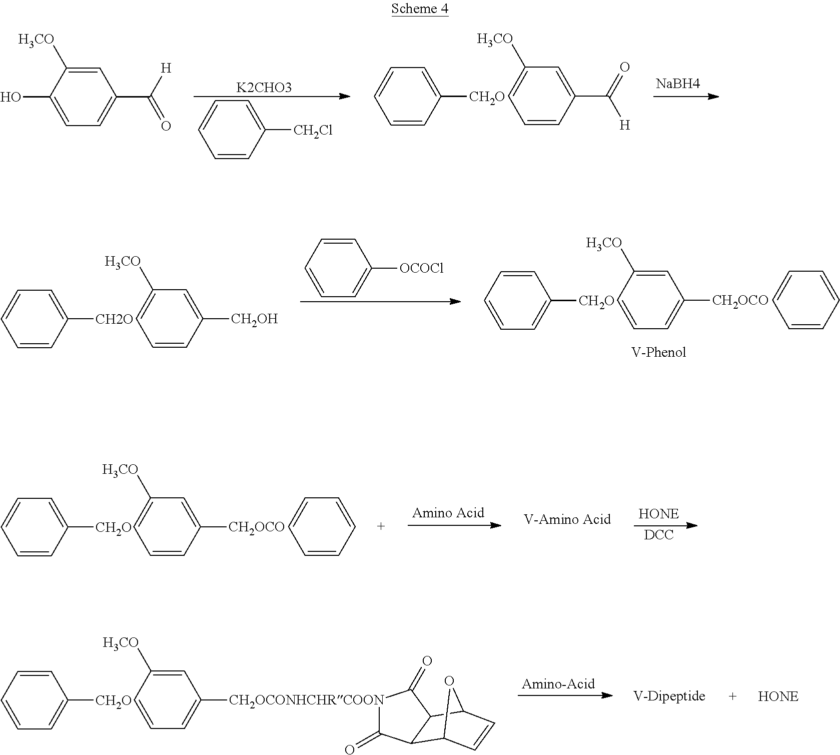

### Peptide Synthesis

The general methods for peptide synthesis, using the new agents, are shown in Scheme 4 and 5

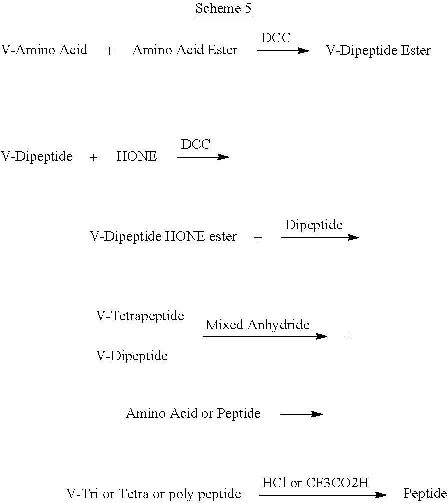

### Removal of Hone and V-phenol

Hone can be removed from an amino acid or a peptide by mixing the product with an aqueous solution that contains an equimolar concentration of ammonium hydroxide or ammonium carbonate and stirred at room temperature. This process results is precipitation of HONE, which can be removed by filtration and the amino acid or peptides recovered as ammonium salts. Scheme 5 shows that before the last step, the synthesized peptides are in V-form. V-phenol is removed by adding concentrated HCl or Trifluoroacetic acid. In this process, V-Phenol is converted to 4-benzyloxy-3-methoxy-benzyl alcohol, which is insoluble in acid medium and can be removed by filtration. Peptides are then recovered by addition of NaHCO3, or NH3OH.

### EXAMPLES

Examples for preparation of various peptides are given below. When dicarboxylic acids such as glutamic or aspartic acids are used, α-carboxylic groups should be free but the second carboxylic groups blocked in the form of methyl or benzyl esters. For the use of diamino acids such as lysine or ornithine, the α-amino groups should be free but the second amino groups are usually in Z-form.

### V-L-Proline

C21H23NO6 MW=385

A mixture of 10 g V-Phenol (0.0275 mole), 2.877 g (0.025 mole) and 3 mL of Tetramethylguanidine in 40 mL of dimethoxyethyleneglycol were stirred at 50° C. for two hours and then overnight at room temperature. To the clear solution, 100 mL water was added and acidified with citric acid. The protected proline was extracted with ethyl acetate. The organic layer was extracted with 50 mL of 0.5 N NaOH. The basic solution was acidified with citric acid. An oil was formed that rapidly solidified into V-L-proline. 9.44 g (98%), which was recrystallized from aqueous isopropanol to snow white micro plates. MP=120-122.

### V-L-Proline-Hone Ester

C9H28N2O9 MW=548

| | | | |
|---|---|---|---|
| V-L-Proline | 9.25 | g | 0.024 mole |
| HONE | 4.34 | g | 0.024 mole |
| DCC | 4.944 | g | 0.024 mole |
| THF | 45 | mL | |

The mixture was stirred for one hour at ice-salt bath temperature and overnight at room temperature. At completion of the reaction, 1 mL 50% acetic acid in water was added and DCU was removed by filtration. Evaporation of THF produced thick oil.

### V-L-Proline-L-Arginine

C27H35N5O MW=541

| | | | |
|---|---|---|---|
| V-L-Proline-HONE-Ester | 4.4 | g | 8 m mole |
| L-Arginine HCI | 1.853 | g | 8.8 m mole |
| Sodium Bicarbonate | 1.478 | g | |
| Dimethoxyethane | 20 | ml | |
| Water | 20 | ml | |

The mixture was stirred overnight at room temperature. Most of the solvent was then evaporated and the product was extracted with chloroform, filtered on celite and chloroform evaporated. A gum was obtained which gradually changed into a power, 4 g, and yield 92%. MP=84-86° C.

### V-L Proline L-Gly-L-Gly-O-Benzyl

C32H35N3O8 MW=589

| | | |
|---|---|---|
| V-L-Proline | 7.70 g | 0.02 mole |
| Gly-Gly-O-Benzyl Tos | 7.82 g | 0.02 mole |
| THF | 50 ml | |
| Et3N | 2.8 ml | |
| DCC | 4.16 g | 0.02 mole |

The mixture was stirred in an ice-salt bath for 1 hour then for 4 hours at room temperature. It was kept at 4° C. overnight. 1 ml of 50% acetic acid in water was then added and DCU was removed by filtration and washed with acetone. An oily residue was produced after evaporation, which gave a precipitate after water was added and re-crystallized from aqueous acetone.

Yield 11.62 g (98.5%) MP=107-110° C.

### V-L-Phenylalanine

C25H25NO6 MW=435

| | | |
|---|---|---|
| L-Phenylalanine | 3.3 g | 0.02 mole |
| V-Phenol | 8.0 g | 0.02 mole |
| Tetramethylguanidine | 2.8 ml | |
| Dimethyformamide | 50 ml | |
| Water | 10 ml | |

The mixture was stirred at 60° C. for 2 hours and then at room temperature overnight. The product was filtered to remove a precipitate, which was identified as an excess of V-Phenol. The clear solution was then extracted with ethyl acetate and the separated aqueous layer was acidified with citric acid after which oil was produced which rapidly turned into 7.3 g solid.

Yield: 85%, MP=145-147° C.

### V-L-Asparagine

C20H22N2O7 MW=402

Prepared as described for V-L Phenylalanine. After completion of the reaction, the solution was acidified with citric acid, extracted with ethyl acetate, and then re-extracted using a NaHCO3 solution. The aqueous phase was acidified again with citric acid to produce a white powder. The yield was 78%. If DMSO was used as the reaction solvent, the yield increased to 90%, MP=136-138° C.

### V-L Asparagine-L-Tyrosine-O-Methyl

C30H33N3O9 MW=579

| | | |
|---|---|---|
| V-Asn | 2.01 g | 5 m mole |
| L-Tyr-OMe | 0.96 g | 5 m mole |
| DCC | 1.03 g | 5 m mole |
| THF | 25 ml | |

Stirred at room temperature for 16 fours, filtered, washed with THF. After evaporation, a gum was produced which was extracted with 1:1 ethyl acetate/petroleum ether to give a crystalline powder. Yield 2.65 g (91%) MP101-103° C.

### V-L-Glycine

C18H19NO6 MW=345

V-L-Gly was prepared as described for V-Phenylalanine, Yield 75%.

### V-L-Glycyl-Glycine

C20 H22N2O7 MW=406

| | | | |
|---|---|---|---|
| Glycyl-Glycine | 2.642 | g | (0.02 mole) |
| V-Phenol | 8.0 | g | (0.02) mole |
| Dimethyformamide | 40 | ml | |
| Tetramethylguanidine | 3 | ml | |
| Water | 10 | ml | |

The mixture was stirred and kept at 60° C. for 24 hours. 100 ml water was added and basified with 3 ml of 2N NaOH. The mixture was extracted with 25 ml Chloroform. The aqueous phase was acidified with Citric Acid to give 3.65 g powder, yield 45%.

### V-Glycyl-Glycine-HONE Ester

C28H27N3O10 MW=565

| | | |
|---|---|---|
| V-Glycyl-Glycine | 3.68 g | 9 m mole |
| HONE | 1.991 g | 9.9 m mole |
| DCC | 2.039 g | 9.9 m mole |
| Acetone | 35 ml | |

The mixture was stirred at ice temperature overnight, filtered and DCU washed with acetone. Acetone evaporation resulted in 3.1 g of a light colored compound. Yield 61%, MP=130-132° C.

### V-Gly-Gly-Leu-Phe

C34H42N4O9 MW=650

| | | | |
|---|---|---|---|
| V-Gly-Gly-HONE | 3.1 | g | 5.5 m mole |
| Leu-Phe | 1.65 | g | 5.5 m mole |
| NaHCO3 | 1.512 | g | 18 m mole |
| Acetone | 25 | ml | |
| Water | 25 | ml | |

The Mixture was stirred at room temperature overnight. Work up as usual. Acidified with citric acid to find a powder.

### Z-L-Leucine-L-Asparagine

C18H25N3O6 MW-379

| | | |
|---|---|---|
| L-Leucine HONE | 27.5 g | 0.66 mole |
| L-Asn | 11 g | 0.66 mole |
| NaHCO3 | | |
| CH3CN | 50 ml | |

NaHCO3 was dissolved in 150 ml of water and added to the mixture, which was stirred at room temperature for 8 hours. 150 ml of water added again. The product was filtered and the filtrate was acidified with citric acid. A solid was formed which was re-crystallized from aqueous isopropanol to give 20 g of colorless small needles. Yield 80% MP=161-163° C.

### Z-L-Leucine-HONE

C22H24N2O6 MW=488

| | | | |
|---|---|---|---|
| Z-L-Leucine | 26.53 | g | 0.1 mole |
| HONE | 18.1 | g | 0.01 mole |
| THF | 75 | ml | |
| CH3CN | 75 | ml | |

The above was cooled in ice bath, then 20.6 g DCC was added and stirred at room temperature overnight. At the end, 2 ml of 50% acetic acid in water was added and after half an hour stirring, DCU was filtered and washed with CH3CN. The solvents were evaporated, leaving 42 g of thick oil. Yield near theory.

### Boc-L-Leucine HONE

C19H26N2O7 MW=394

| | | |
|---|---|---|
| Boc-L-Leucine | 18 g | 0.0722 mole |
| HONE | 13 g | 0.0722 mole |
| THF | 150 ml | |
| DCC | 14.87 g | 0.0722 mole |

The mixture stirred for 2 hours at ice-salt water temperature, then overnight at room temperature. Work-up resulted in 26.8 g of a white crystal. Yield 94.4%, MP=141-143° C.

### Boc-L-Leucine-L-Leucine

C17H32N2O5 MW=349

| | | | |
|---|---|---|---|
| Boc-L-Leucine HONE | 3.49 | g | 0.01 mole |
| L-Leucine | 1.317 | g | 0.01 mole |
| NaHCO3 | 2.10 | g | 0.025 mole |
| DMF | 30 | ml | |
| Water | 15 | ml | |

Stirred for 6 hours then 25 ml more water is added and stirring continued overnight. The product was filtered and the solid was washed with water, cooled and acidified with citric acid to give 2.62 g (76%) of crystal. MP=71-73° C.

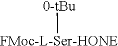

C30H30N2O8 MW=546

| | | |
|---|---|---|
| 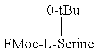 | 3.84 g | 0.01 mole |
| HONE | 1.82 g | 0.01 mole |
| THF | 50 ml | |
| DCC | 2.1 g | |

The mixture was stirred at room temperature overnight, then, 0.5 ml 50% acetic acid was added and filtered to remove the insoluble DCU (washed with acetone). The filtrate was evaporated resulting in a solid, which was dissolved in warm acetone. After cooling and by addition of petroleum ether, a colorless precipitate was formed which crystallized after evaporation. Yield 5.4 g, 100%, MP=110-112° C.

### V-Beta Alanine

C19H21NO6, MW=359

It was prepared as described for V-L-Alanine, giving a crystal powder.

Yield 84.4%, MP=111-113° C.

### V-L-Methionine

C21H35NO6S MW=419

| | | | |
|---|---|---|---|
| V-Phenol | 21.80 | g | 0.06 mole |
| L-Methionine | 8.95 | g | 0.06 mole |
| Tetramethylguanidine | 7.65 | ml | |
| Dimethyformamide | 80 | ml | |
| Water | 20 | ml | |

Stirred for 3 hours at 60° C. then overnight at room temperature. At the end, 100 ml of water was added, the solution was charcoaled and acidified with citric acid, following which oil was formed and was extracted with 30 ml ethyl acetate. V-Met was extracted with 0.05N NaOH, acidified with citric acid and a crystalline compound was recovered. Yield 19.35 g (77%).

### V-L-Methionine-L-Phenylalanine

C30H34N2O7S MW=566

| | | | |
|---|---|---|---|
| V-L-Methionine-Hone | 4.78 | g | 8.2 m mole |
| L-Phenylalanine | 1.5 | g | 9.0 m mole |
| NaHCO3 | 2.3 | g | 27.0 m mole |
| Acetone | 25 | ml | |
| Water | 25 | ml | |

The Mixture was stirred at room temperature overnight, and at the end filtered, the acidified with citric acid, producing a white powder. Yield 3.95 g (85%).

### V-L-Methionine-L-Tyrosine Methyl Ester

C31H36N2O8S MW=596

| | | | |
|---|---|---|---|
| V-L-Methionine | 6.072 | g | 0.015 mole |
| L-Tyrosine-Methyl Ester HCL | 3.23 | g | 0.015 mole |
| DCC | 3.09 | g | 0.015 mole |
| Triethylamine | 2.31 | ml | |
| THF | 50 | ml | |

Stirred for 2 hours at ice-salt bath temperature then overnight at room temperature. The work-up produced an oil, which turned into a solid form after it was kept for 24 hours under vacuum. Yield 7.8 g (95%) MP=119-121° C. The compound was re-crystallized in isopropanol.

### V-Methionine-Proline-Phenylalanine

C35H41N3O8 MW=663

V-Methionine was reacted with Proline-Phenylalanine using a mixed anhydride method. A transparent gum was produced. Yield: 100%.

### Deprotection of V-Tripeptide

The above V-Tripeptide was treated with 10 ml of concentrated HCl for 10 minutes, generating bubbling CO2 and V-Chloride. The mixture was filtered and neutralized with Amonia. Evaporation resulted in a white crystalline powder, with strong methionine odor. The yield was 65%, MP=>300° C.

### V-Sarcosine

C19H21NO6, MW=309

| | |
|---|---|
| L-Sarcosine | 0.89 g |
| V-Phenol | 4.0 g |
| Dimethylformamide | 20.0 ml |
| Water | 4.0 ml |
| Tetramethylguanidine | 1.4 ml |

The mixture stirred at room temperature for 6 hours, and then 80 ml water was added and acidified with citric acid. A crystalline compound was produced, 3.23 g, yield=90%. The product was re-crystallized from isopropanol. MP=133-134° C.

Synthesis of Ala-Ser-Thr-Thr-Thr-Asn-Tyr-Thr. CF3COOH Known as T4 Octapeptide, an HIV-Receptor Inhibitor

C37H56F3N9O18 MW=971
1) L-Ala→V-L-Ala→V-Ala-HONE→V-Ala-Ser
2) V-Ala-Ser→V-Ala-Ser-HONE→V-Ala-Ser-Thr
3) L-Thr→V-Thr→V-Thr-HONE→V-Thr-Thr
4) L-Asn→V-Asn→V-Asn-HONE→V-Asn-Tyr
5) V-Asn-Tyr→V-Asn-Tyr-Thr-OMe (by mixed anhydride reaction)
6) V-Asn-Tyr-Thr-OMe→Asn-Tyr-Thr-OMe.HCI
7) V-Thr-Thr (#3 above)→V-Thr-Thr-HONE→V-Thr-Thr-Asn-Tyr-Thr-OMe
8) V-Thr-Thr-Asn-Tyr-Thr-OMe→Thr-Thr-Asn-Tyr-Thr-OMe
9) V-Ala-Ser-Thr+Thr-Thr-Asn-Tyr-Thr-OMe (by DCC reaction)→V-Ala-Ser-Thr-Thr-Thr-Asn-Tyr-Thr-OMe (yield 80%)
10) Saponified→V-Ala-Ser-Thr-Thr-Thr-Asn-Tyr-Thr. CF3COOH (yield 50%)

The above was cooled in ice bath, then 20.6 g DCC was added and stirred at room temperature overnight. At the end, 2 ml of 50% acetic acid in water was added and after half an hour stirring, DCU was filtered and washed with CH3CN. The solvents were evaporated, leaving 42 g of thick oil. Yield near theory. The structure was confirmed by amino acid analysis. MP=165-167° C.

1: The novel composition of matter and method for synthesis of V-Phenol, 4-Benzyloxy-3-Methoxybenzyloxy-Phenoxy Carbonate, a newly developed vanillin derivative, as an amino acid N-protecting agent:
Synthesis of V-Phenol (as shown in Scheme 1)

| | |
|---|---|
| Vanillin 136.8 g | (1 Mole) |
| Benzyl Chloride 114 ml | (1.1 Mole) |
| Anhydrous Potassium Carbonate 63 g | (0.456 Mole) |
| Isopropanol 450 ml | |

The mixture is refluxed for 18 hours. To the hot mixture 24 g of Sodium Borohydride is added slowly (to keep the reaction under control). Gradually a semisolid mass is formed, which is kept under reflux for additional 2 hours. After cooling, 1,500 ml cold water is added and stirred to give a crystalline colorless compound: 190 g, (83% yield), MP 62-63° C. The structure is confirmed to be 4-benzyloxy-3-methoxy-benzyl alcohol. The alcohol obtained is dried and dissolved in 1140 ml CH2Cl2 and 100 ml pyridine is added. The mixture is cooled in an ice/salt bath for half an hour and then gradually 125 mL phenylchloroformate is added. After 2 hours of stirring at room temperature, cold water is added to give a crystalline compound after removal of most of CH2Cl2. The preparation is filtered and re-crystallized from toluene to give 299 g of white crystals (82% yield), MP: 118-120° C.

2: The use of V-Phenol as an amino acid N-protecting agent and novel N-protected amino acids comprising new compositions of matter produced by V-Phenol reacting with any amino acids in mild alkaline solutions in various organic solvents such as shown in Scheme 2.

3. The use set forth in claim 2 wherein the amino acids are L amino acids.

4. The use set forth in claim 2 wherein the amino acids are D amino acids.

5. The use set forth in claim 2 wherein the amino acids are physiologically active.

6. The use set forth in claim 2 wherein the amino acids are physiologically inactive.

7. The use set forth in claim 2 wherein the amino acid side chains contain one or more basic groups.

8. The use set forth in claim 2 wherein the amino acid side chains contain one or more acidic groups.

9. The use set forth in claim 2 wherein the amino acid side chains contain one or more aromatic rings.

10: Method of synthesis and novel composition of matter of HONE, exo-N-hydroxy-7-oxabycyclo[2.2.1]hept-5-ene-2,3-dicarboximide, a new C-protecting agent for peptide synthesis. The method comprises:
a) 80 g maleic anhydride (shown in Scheme 3) (2 moles) is dissolved in 350 ml toluene by stirring and gentle warming, cooled to room temperature and then 120 ml of furan is gradually added. After cooling, a crystalline compound (anhydride) is formed. 100% yield. MP=118-120° C.
b) To synthesize HONE a mixture of 320 g anhydride (2 mole), 168 g sodium bicarbonate (2 mole) or equivalent Sodium Acetate, 139 g hydroxylamine hydrochloride and 400 ml of water is warmed at 60-70° C. for one hour and then cooled. Crystals are formed, which are filtered by suction and washed with 50 mL cold water. After drying, 350 g (97%) crystalline compound is obtained. Re-crystallization is done in boiling water to give prisms. MP=202-204° C.

11: Method of Preparation of, and novel compositions of matter of N-protected Amino acid Active Esters utilizing HONE as shown in Scheme 4.
N-protected amino acids and HONE are dissolved in non-protic solvents and reacted with dicyclohexylcarbodiimide (DCC) at cold to give N-protected amino acid-HONE esters which are novel compositions of matter.

12: Use of V-Phenol N-protected amino groups for peptide synthesis as shown in Scheme 5. Hone is removed from an amino acid or a peptide by mixing the product with an aqueous solution that contains an equimolar concentration of ammonium hydroxide or ammonium carbonate and stirred at room temperature. This process results is precipitation of HONE, which can be removed by filtration and the amino acid or peptides recovered as ammonium salts. Before the last step, the synthesized peptides are in V-form. V-phenol is removed by adding concentrated HCl or Trifluoroacetic acid. In this process, V-Phenol is converted to 4-benzyloxy-3-methoxy-benzyl alcohol, which is insoluble in acid medium and can be removed by filtration. Peptides are then recovered by addition of NaHCO3, or NH3OH.

13: The use set forth in claim 12 wherein the peptides being synthesized contain from two to twenty amino acids.

14. The use set forth in claim 12 wherein L-amino acids are used.

15. The use set forth in claim 12 wherein D-amino acids are used.

16. The use set forth in claim 12 wherein the amino acids used are physiologically active.

17. The use set forth in claim 12 wherein the amino acids are non-physiological.

18. The use set forth in claim 12 wherein the amino acid side chains contain one or more basic groups.

19. The use set forth in claim 12 wherein the amino acid side chains contain one or more acidic groups.

20. The use set forth in claim 12 wherein the amino acid side chains contain one or more aromatic rings.

21: The use set forth in claim 12 wherein the amino acids used to synthesize peptides are C-protected by HONE or by methyl, ethyl, benzyl, or other esters. that are removable by sodium hydroxide followed by acidification to remove sodium by any acid.

22: The use set forth in claim 12 wherein the amino acids used to synthesize peptides are C-protected by benzyl, methyl, allyl, tButyl or paramethoxy benzyl or similar esters.

23: The use set forth in claim 12 wherein a new amino acid is added to an existing polypeptide or other compounds that contain carboxylic acids.

24. The use of C-protected by HONE amino acids for peptide synthesis.

25. The use of claim 23 wherein the peptide being synthesized contains from two to twenty amino acids.

26. The use set forth in claim 23 wherein the amino group is protected by V-Phenol.

27. The use set forth in claim 23 wherein the amino group is protected by other protecting agents such as Cbz, Boc, Fmoc, Truc, or similar agents.

28. The use set forth in claim 23 wherein a HONE protected amino acid is used for addition of any amino acid to a pre-existing peptide or protein.

29. The use set forth in claim 23 wherein the HONE protected amino acids are used for the addition of the amino group of any amino acid to fatty acids and other carboxylic acid-containing structures.

30. The use of V-Phenol protected amino acids for peptide synthesis employing liquid phase, Merrifield's type of solid phase or microwave-assisted methods.

31. The use of HONE protected amino acids for peptide synthesis employing liquid phase, Merrifield's type of solid phase or microwave assisted methods.
